(19) United States
(12) Patent Application Publication
(10) Pub. No.: US 2010/0003351 A1
(43) Pub. Date: Jan. 7, 2010

(54) COMPOSITIONS COMPRISING RHODIOLA ROSEA AND METHODS OF USE THEREOF

(76) Inventor: Russell M. JAFFE, Sterling, VA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/166,825

(22) Filed: Jul. 2, 2008

(51) Int. Cl. A61K 36/00; A61K 31/70

(52) U.S. Cl. 424/725; 514/27

(57) ABSTRACT

The present invention relates to compositions and kits comprising an extract of Rhodiola rosea, and one or more extracts selected from the group consisting of Magnolia officinalis and Phellodendron amurense. The present invention also relates to methods of treating a disease or condition associated with adrenal fatigue and/or insulin resistance by administering these compositions.

### FIELD OF THE INVENTION

This invention relates to compositions comprising an extract of Rhodiola rosea, and one or more extracts selected from the group consisting of Magnolia officinalis and Phellodendron amurense. The invention also relates to methods of treating a disease or condition associated with adrenal fatigue and/or insulin resistance by administering these compositions.

### BACKGROUND OF THE INVENTION

Adrenal fatigue is prevalent in today's society, though it was first described in textbooks over a century ago. The term refers to exhaustion of adrenal glands, which secrete two hormones related to stress levels: cortisol (stress-response hormone) and dehydroepiandrosterone (DHEA) (mother hormone or “feel good” hormone). Affected individuals have elevated cortisol levels and depressed DHEA levels, and may suffer from a broad spectrum of non-specific yet debilitating symptoms, such as low energy, sleep problems, weight gain, memory loss, and susceptibility to infections.

Combined extracts of Magnolia officinalis and Phellodendron amurense (commercially available under the tradename Relora®) have been shown in clinical studies to improve cortisol and DHEA levels in the body and also to curb stress-related eating. However, side effects can limit the use of such extracts (e.g., Garrison et al., Alternative Therapies in Health and Medicine 2006, 12(1): 50-54). Hence there is a need for compositions which improve the delivery and/or reduce the side effects of these medicinal extracts.

Many other individuals suffer from depressed or irregular cortisol levels. Cortisol is a “fight or flight” stress hormone and powerful defense agent. It heightens alertness, slows down digestion, suppresses immune response when needed (e.g., allergies or excessive inflammatory response), and maintains healthy energy levels in the body. Morning cortisol levels are particularly important for sufficient energy to start the day. Individuals with depressed morning cortisol levels have difficulty concentrating and functioning during morning hours. Hence, there is a need for a natural medicinal agent that modulates cortisol levels: lowering, raising, or regulating the natural daily rhythm of cortisol levels, according to the affected individual's needs.

In addition, there is a need for natural medicinal sources to more broadly address the conditions associated with adrenal fatigue, rather than simply adjust cortisol and DHEA levels. Adrenal function, blood sugar levels, and our internal stress levels are intimately related. Low cortisol levels lead to slow energy production, and high insulin levels lead to increased glucose demand. In order to prevent too much glucose from flooding cells, insulin resistance develops, often followed by adult-onset (Type 2) diabetes. Thus those suffering from insulin resistance and/or Type 2 diabetes (in addition to adrenal fatigue) could benefit greatly from a medicinal agent that restores glucose-insulin balance.

Therefore, there is a need for a medicinal agent that can treat the symptoms and hormonal imbalances associated with adrenal fatigue, tailored to an individual's needs, as well as suppress insulin resistance and restore a healthy glucose-insulin balance.

### SUMMARY OF THE INVENTION

In one embodiment, the present invention is directed to a composition comprising an extract of Rhodiola rosea and one or more extracts selected from the group consisting of Magnolia officinalis and Phellodendron amurense.

In another embodiment, the present invention is directed to a method for treating a disease or condition associated with adrenal fatigue comprising administering to a mammal an effective amount of a composition comprising an extract of Rhodiola rosea, and one or more extracts selected from the group consisting of Magnolia officinalis and Phellodendron amurense.

In yet another embodiment, the present invention is directed to a kit for treating a disease or condition associated with adrenal fatigue and/or insulin resistance comprising a container comprising an extract of Rhodiola rosea; one or more plant extracts selected from the group consisting of Magnolia officinalis and Phellodendron amurense; an oil, medium chain triglycerides; optionally one or more ingredients selected from the group consisting of phosphatidylcholine, beeswax, gelatin, glycerine, water, and turmeric; and instructions for use.

### BRIEF DESCRIPTION OF THE DRAWINGS

FIG. 1 shows the average values of salivary cortisol (ng/mL) of 25 subjects in a six-week clinical study in which the composition of Example 1 was administered as 2 softgel capsules daily. The subjects' cortisol levels were measured in saliva samples taken at 7:00 am, 11:00 am, 5:00 pm, and 10:00 pm on two days: pre-treatment (“Pre) and post-treatment (“Post”).

FIG. 2 shows the values of cortisol in saliva samples of 2 subjects (Cases 1 and 2) with elevated first-morning cortisol levels in that same clinical study. The subjects' cortisol levels were measured in saliva samples taken at 7:00 am, 11:00 am, 5:00 pm, and 10:00 pm on two days: pre-treatment (“Pre) and post-treatment (“Post”).

FIGS. 3 and 4 show the glucose/insulin (GI) ratios and homeostatic model assessment (HOMA) values for two subjects with insulin resistance before (“Pre”) and after (“Post”) six weeks' treatment with the composition of Example 1 (2 softgels administered daily). The GI ratio and HOMA values are measurements of insulin resistance.

### DETAILED DESCRIPTION OF THE INVENTION

All documents cited herein are incorporated by reference in their entirety for all purposes.

In its various embodiments, the present invention relates to compositions and kits comprising an extract of Rhodiola rosea, and one or more extracts selected from the group consisting of Magnolia officinalis and Phellodendron amurense. The present invention also relates to methods of treating a disease or condition associated with adrenal fatigue and/or insulin resistance by administering these compositions.

Extract of Rhodiola rosea

Rhodiola rosea is one of 200 species of plants falling within the genus Rhodiola in the Crassulaceae family. Rhodiola rosea is a plant endogenous to the high altitudes of the Artic and mountainous regions of Europe and Asia. It has been used as a traditional medicine in Eastern Europe and Asia for its adaptogenic, CNS-stimulating, cardioprotective, anticancer, antioxidant, and/or antimicrobial effects. Some 20 species in the Rhodiola genus have been used as traditional medicines. Rhodiola rosea, its characteristics, and its therapeutic effects have been described; e.g., Kelly, G S, Alter. Med. Rev. 2001, 6(3), 293-302; Krajewska-Patan, Herba Polonica 2007, 53(4), 77-87; US Pub. No. US 2006/0002871, each of which is herein incorporated by reference in its entirety for all purposes.

Alternative names for Rhodiola rosea include “golden root,” “Arctic root,” “Crenulin,” (common names), and Sedum rosea, Sedum rosea var. roanense, Sedum roseum, and Sedum rhodiola (Latin names).

Rhodiola rosea has been recognized for its adaptogenic properties. As used herein, the term “adaptogen” refers to an agent that allows an organism to counteract adverse physical, chemical, or biological stressors by generating non-specific resistance. Under this definition, an adaptogen meets three criteria: (i) produces a non-specific response in an organism, i.e., an increase in power of resistance against physical, chemical, and biological stressors; (ii) normalizes influence on physiology, regardless of the direction of change from physiological norms caused by the stressor; and (iii) does not influence normal physiological functions more than is required to provide non-specific resistance.

A number of chemical compounds of widely diverse chemical structures have been isolated from the root and above-ground parts of Rhodiola rosea plants. Some of these compounds show antioxidative, cardioprotective, adaptogenic, or other therapeutic effects. Compounds isolated from the root include fatty acids, flavonoids, tannins, simple phenolic derivatives (e.g., p-tyrosol), phenolic glycosides (e.g., salidroside (also called rhodioloside)), phenylpropanoids (e.g., rosavin, rosarin, rosin), flavonoids (e.g., rodionin, rodiolin, rhodiosin, acetylrodalgin, and tricin), phenolic acids (e.g., gallic acid, chlorogenic acid, hydroxycinnamic acid), monoterpenes, sterols (e.g., β-sitosterol, dukosterol), and essential oils. Adaptogenic and CNS-stimulating properties have been attributed to salidroside, tyrosol, and phenylpropanoids.

As used in the art and herein, the term “rosavin” refers to a single compound, a particular cinnamyl alcohol glucoside (arabinose-glucose-O-cinnamyl). As used herein and in the art, “rosavins” refers to three compounds from the root of Rhodiola rosea: rosin, rosavin (as defined herein), and rosarin, all of which share a glucose-O-cinnamyl component. “Rosavin™” is also used as a tradename for a particular Siberian Rhodiola rosea product.

Some compounds (e.g., p-tyrosol) reliably appear in all studied species of Rhodiola, but many of the glycosides have not been found in all studied Rhodiola species. Furthermore, the proportions of certain compounds in the plant (e.g., rosavin, rosin, rosarin, and salidroside) vary with the season and region of growth. See Kucinskaite et al., Medicina (Kaunas) 2007, 43(6), 487-494. Due to this variability in the Rhodiola genus, analysis by high performance liquid chromatography (HPLC) is used to determine the glycoside content of a particular Rhodiola extract. Rosavin or salidroside are the usual constituents selected for standardization of Rhodiola rosea extracts.

Rhodiola rosea extracts are obtained by contacting the plant or parts of the plant (e.g., root, leaves, etc., optionally crushed or chopped) with a suitable solvent, such as water, alcohol, supercritical CO₂, or any other solvent, or mixture of solvents capable of dissolving component(s) of the plant or plant part. The choice of the solvent may be made e.g., based on the properties of the active ingredient that is to be extracted or concentrated by the solvent. Preferred active ingredients of Rhodiola rosea include but are not limited to, rosavins, salidroside and tyrosol. These ingredients can be extracted in the same step, e.g., using an alcoholic, aqueous solvent, or supercritical CO₂, or they may be extracted individually, each time using a solvent which is especially effective for extracting the particular target ingredient from the plant. Extracts of Rhodiola rosea are commercially available; e.g., from Nature's Thyme, LLC, East Hanover, N.J.

The proportions or presence of compounds in Rhodiola rosea can vary due to their presence (or absence) in the plant and the extraction method used, particularly for rosavins. Some extracts may contain very little or no detectable amounts of rosavins. Techniques for optimizing extracted amounts of the compounds in Rhodiola rosea are known in the art. Although compounds of known therapeutic activity may be the principle active components of the Rhodiola rosea extract (e.g., salidroside, rosavins, etc.), other compounds may be present that may contribute to the extract's therapeutic effects.

The concentration of an ingredient in the extracts used in the compositions of the present invention is expressed as the percentage (%) of that ingredient by weight of the total extract. The concentration of an ingredient in the compositions of the present invention is expressed as % of that ingredient by weight of the total composition.

In one embodiment, the extract of Rhodiola rosea used in the compositions of the present invention contains rosavins. In another embodiment, the extract contains one or more compounds selected from the group consisting of rosavin, rosin, and rosarin. In another embodiment, the rosavins are present in the extract in the amount of from about 0.5% to about 50%, or from about 1% to about 20%, or from about 1% to about 10%, including about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, or about 50%, inclusive of all ranges and subranges therebetween. In another embodiment, rosavins are present in the extract of the compositions of the present invention at levels of at least about 3%; in yet another embodiment, rosavins are present at levels of about 3%.

In one embodiment, the extract of Rhodiola rosea contains salidroside. In another embodiment, salidroside is present in the extract at levels ranging from about 0.1% to about 20%, or from about 0.5% to about 10%, or from about 0.5% to about 5%, including about 0.1%, about 0.15%, about 0.2%, about 0.25%, about 0.3%, about 0.35%, about 0.4%, about 0.45%, about 0.5%, about 0.6%, about 0.65%, about 0.7%, about 0.75%, about 0.8%, about 0.85%, about 0.9%, about 0.95%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, or about 20%, inclusive of all ranges and subranges therebetween. In another embodiment, salidroside is present in the extract of the compositions of the present invention at levels of at least about 1%; in yet another embodiment, salidroside is present at levels of about 1%.

In one embodiment, the extract of Rhodiola rosea used in the compositions of the present invention contains a combination of rosavins and salidroside. In another embodiment, the extract contains about 0.5-50% rosavins and about 0.5%-20% salidroside. In another embodiment, the extract contains about 1-10% rosavins and about 0.5-10% salidroside. In another embodiment, the extract contains at least about 3% rosavins and at least about 1% salidroside. In another embodiment, the extract contains about 3% rosavins and about 1% salidroside.

In another embodiment, the extract of Rhodiola rosea used in the compositions of the present invention contains a combination of rosavins and salidroside in which the ratio of rosavins to salidroside ranges from about 1:10 to about 10:1, including from about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, or about 1:1, inclusive of all ranges and subranges therebetween. In yet another embodiment, the ratio of rosavins to salidroside is about 10:1, about 9:1, about 8:1, about 7:1, about 6:1, about 5:1, about 3:1, about 2:1, about 1:1, or about 0.5:1. In yet another embodiment, the ratio of rosavins to salidroside is at least about 3:1. In another embodiment, the ratio of rosavins to salidroside is about 3:1

The amount of an extract of Rhodiola rosea in the compositions of the present invention can vary, depending on the concentration of active components in the extract and the desired concentration of active components in the final composition. In one embodiment, the extract of Rhodiola rosea is present in the compositions of the present invention in the amount of about 0.1% to about 90%, about 1% to about 50%, about 2% to about 20%, or about 10% to about 15% by weight of the total composition, inclusive of all ranges and subranges therebetween. In another embodiment, the extract of Rhodiola rosea is present in the amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

Extracts of Magnolia officinalis and Phellodendron amurense
Magnolia officinalis

In one embodiment, the compositions of the present invention comprise an extract of Rhodiola rosea and an extract of Magnolia officinalis.

The properties and composition of extracts of Magnolia officinalis have been described. See, e.g., U.S. Pat. No. 6,582,735, herein incorporated by reference in its entirety for all purposes. Magnolia officinalis is one species falling within the genus Magnolia and the family Magnoliaceae. There are two subvarieties of the Magnolia officinalis plant: Magnolia officinalis var. biloba and Magnolia officinalis var. officinalis. Alternative names for Magnolia officinalis are Magnolia bark, houpo or hou po. Magnolia officinalis has been reported to have a number of medicinal properties, primarily attributed to the chemical compounds magnolol, honokio, and/or magnofluorine.

In one embodiment of the compositions of the present invention, an extract of Magnolia officinalis is present in an amount of about 5% to about 20%, about 7% to about 19%, or about 8% to about 16% by weight relative to the weight of the total composition.

Phellodendron amurense

In another embodiment, the compositions of the present invention comprise an extract of Rhodiola rosea and an extract of Phellodendron amurense. The properties and composition of extracts of Phellodendron amurense have been described. See, e.g., Publ. No. US 2003/0215531. Phellodendron amurense derives from the Amur corktree and falls within the genus Phellodendron and the family Rutaceae. Alternative names for Phellodendron amurense include Amur corktree, Chinese corktree, phellodendron bark, huang bai, huang po, kihada (common names), and Phellodendron japonicum, Phellodendron lavalleei, Phellodendron sachalinense, and Phellodendron wilsonii (Latin names). The bark of Phellodendron amurense is reported to have many medicinal properties, including, for example, antibacterial antirheumatic, aphrodisiac, and CNS stimulatory activities.

In one embodiment of the compositions of the present invention, an extract of Phellodendron amurense is present in an amount of about 5% to about 20%, about 7% to about 19%, or about 8% to about 16% by weight relative to the weight of the total composition.

Extracts of Magnolia officinalis and Phellodendron amurense are commercially available, e.g., from Next Pharmaceuticals, Salinas, Calif., sold together in a single product under the registered trademark Relora®. The administration of Relora® has been shown in clinical studies to lower cortisol levels and increase DHEA levels in the body and also to alleviate stress-related eating. However, side effects can offset its benefits (e.g., Garrison et al., Alternative Therapies in Health and Medicine 2006, 12(1):50-54).

In one embodiment of the compositions of the present invention, the extracts of Magnolia officinalis and Phellodendron amurense are prepared using supercritical fluid extraction. In another embodiment, carbon dioxide serves as the supercritical fluid. Supercritical fluid extraction has certain advantages over traditional liquid extraction, including, for example, selectivity for extracting certain compounds while minimizing impurities, improved purification, and elimination of residual solvents.

In another embodiment, extracts of Magnolia officinalis and Phellodendron amurense may be prepared by contacting the plant or parts of the plant (e.g., optionally crushed or chopped) with a suitable solvent (.e.g, water, alcohol, or any other solvent, or mixture of solvents capable of dissolving component(s) of the plant or plant part). The choice of solvent may be made routinely by one skilled in the art, e.g., based on the properties of the active ingredient(s) that is to be extracted or concentrated by the solvent. Preferred solvents are water and lower alcohol. These ingredients may be extracted in one step, or individually, each time using a solvent which is especially effective for extracting that particular target ingredient from the plant.

Extracts of Rhodiola rosea, Magnolia officincals, and Phellodendron amurense

In one embodiment, the compositions of the present invention comprise an extract of Rhodiola rosea, an extract of Magnolia officinalis, and an extract of Phellodendron amurense. When extracts of Magnolia officinalis and Phellodendron amurense are both present in the compositions of the present invention, the ratio of the extract of Magnolia officinalis to the extract of Phellodendron amurense ranges from about 1:10 to about 10:1, including from about 1:9 to about 9:1, about 1:8 to about 8: 1, about 1:7 to about 7:1, about 1:6 to about 6:1, about 1:5 to about 5:1, about 1:4 to about 4:1, about 1:3 to about 3:1, about 1:2 to about 2:1, or about 1:1, inclusive of all ranges and subranges therebetween. In another embodiment, when both extracts are present, each extract is present in the compositions of the present invention in an amount of from about 1% to about 19%, about 2% to about 15%, about 5% to about 12%, or about 10% by weight relative to the total weight of the composition.

In yet another embodiment of the compositions of the present invention, the ratio by weight of the extract(s) of Rhodiola rosea to the combined extracts of Magnolia officinalis and Phellodendron amurense ranges from about 2:5 to about 4:5, about 2.5 to about 3:5, or about 3:5 to about 4:5, inclusive of all ranges and subranges therebetween. In another embodiment, the ratio by weight of the extract(s) of Rhodiola rosea to the combined extracts of Magnolia officinalis and Phellodendron amurense is about 2:5, about 3:5, or about 4:5.

The relative amounts of the extracts of Rhodiola rosea, Magnolia officinalis, and Phellodendron amurense contribute to the biological activity of the compositions of the present invention. All of these extracts have the potential to be stimulatory or inhibitory. Their combination in appropriate amounts provides the desired effects on cortisol and/or DHEA levels, glucose/insulin balance, and symptoms related to adrenal fatigue and/or insulin resistance. Too much of any one extract relative to the others may not improve such clinical effects, and, in fact, may aggravate them.

Those skilled in the art will recognize that extracts of the same plant material obtained under different conditions (e.g. solvent, extraction temperature, and/or extraction duration, etc.) would have somewhat different compositions. For example, extraction with more polar solvents would result in extracts containing the more polar extractable components of the plant material, whereas extraction with less polar solvents would provide extracts containing higher proportions of the less polar extractable components. Accordingly, the compositions of the present invention can comprise one or more different extracts of Rhodiola rosea, Magnolia officinalis, and/or Phellodendron amurense.

### Oil

In one embodiment, the compositions of the present invention comprise one or more oils. Non-limiting examples of oils suitable for use in the compositions of the present invention include oils suitable for human consumption such as petroleum oils (e.g., mineral oil, paraffin oil, etc.); animal oils; vegetable oils, including seed oils and nut oils; modified oils (e.g. hydrogenated or partially hydrogenated vegetable oils); or synthetic oils.

In another embodiment, the compositions of the present invention comprise one or more oils containing a substantial amount of omega-3 fatty acids, including, for example, fish oil (e.g., from salmon, mackerel, sardines, anchovies, albacore tuna, cod liver oil, or mixtures thereof), flaxseed (or linseed) oil, chia seed oil, hempseed oil, walnut oil, pumpkin seed oil, Brazilian nut oil, butternut oil, pecan oil, hazelnut oil, sesame seed oil, avocado oil, acai palm oil, canola (rapeseed) oil, soybean oil, kiwi fruit seed oil, perilla seed oil, lingonberry seed oil, camelina seed oil, purslane seed oil, black raspberry seed oil, sea buckthorn seed oil, or mixtures thereof.

In yet another embodiment, the compositions of the present invention comprise one or more oils containing a substantial amount of alpha-linolenic acid (ALA), including, for example, flaxseed (or linseed oil), chia seed oil, perilla seed oil, lingonberry seed oil, camelina seed oil, purslane seed oil, black raspberry seed oil, sea buckthorn seed oil, canola (rapeseed) oil, or soybean oil. In another embodiment, the compositions comprise one or more oils containing an ALA content of about 5-85%, about 10-75%, about 20%-70%, about 30-65%, or about 40-60%, inclusive of all ranges and subranges therebetween, and including about 5%, about 10%, about 15%, about 20%, about 25%, about 30%, about 35%, about 40%, about 45%, about 50%, about 55%, about 60%, about 65%, about 70%, about 75%, about 80%, or about 85%.

In a particular embodiment, the compositions of the present invention comprise perilla seed oil. In another embodiment, the perilla seed oil is derived from Perilla frutescens seeds. Perilla seed oil is rich in omega-3 fatty acids and has an ALA content of about 50-60%. Perilla seed oil is desirable to include in the compositions of the present invention because it is plant-based (appealing to vegetarians) and it is an alternative to flaxseed oil (another plant-based oil high in ALA) which is more common in foods and may elicit sensitivity responses in certain individuals.

ALA is an omega-3 fatty acid, which is converted to eicosapentaenoic acid (EPA) and docosahexaenoic acid (DHA) in the body. ALA has been indicated as having anti-inflammatory, cardioprotective, and anti-cancer properties.

In one embodiment, the compositions of the present invention have a total oil content (of one or more oils) of about 5-90%, about 10-80%, about 15-70%, about 20-60%, about 25-50%, 30-45%, or about 35-40%, inclusive of all ranges and subranges therebetween, and including about 5%, about 10%, about 15%, about 20%, about 25%, about 30%, about 35%, about 40%, about 45%, about 50%, about 55%, about 60%, about 65%, about 70%, about 75%, about 80%, about 85%, or about 90%.

In one embodiment, the compositions of the present invention comprise an extract of Rhodiola rosea, one or more extracts selected from the group consisting of Magnolia officinalis and Phellodendron amurense, and at least one oil. In another embodiment, the compositions comprise an extract of Rhodiola rosea, an extract of Magnolia officinalis, and at least one oil. In another embodiment, the compositions comprise an extract of Rhodiola rosea, an extract of Phellodendron amurense, and at least one oil. In yet another embodiment, the compositions comprise an extract of Rhodiola rosea, an extract of Magnolia officinalis, an extract of Phellodendron amurense, and at least one oil.

### Medium Chain Triglycerides

As used here, the term “medium chain triglycerides” (“MCTs”) refers to fatty acid esters of glyercol where the fatty acids have about 6 to about 12 carbon atoms in the fatty chain. Suitable MCTs include any medium chain triglyceride or mixture of medium chain triglycerides suitable for human consumption. In one embodiment, the medium chain triglycerides are derived from raw palm fruit. Coconut oil and palm kernel oils can also be used as sources of MCTs. MCTs include a pure medium chain triglyceride or a mixture of different medium chain triglycerides.

In one embodiment, the compositions of the present invention comprise an extract of Rhodiola rosea, one or more extracts selected from the group consisting of Magnolia officinalis and Phellodendron amurense, at least one oil, and MCTs. In another embodiment, the compositions comprise an extract of Rhodiola rosea, an extract of Magnolia officinalis, at least one oil, and MCTs. In another embodiment, the compositions comprise an extract of Rhodiola rosea, an extract of Phellodendron amurense, at least one oil, and MCTs. In yet another embodiment, the compositions comprise an extract of Rhodiola rosea, an extract of Magnolia officinalis, an extract of Phellodendron amurense, at least one oil, and MCTs.

In one embodiment of the compositions of the present invention, MCTs are present in an amount of about 1% to about 50%, about 5% to about 25%, or about 10% to about 15%, inclusive of all ranges and subranges therebetween. In one embodiment, the compositions of the present invention contain about 10% of MCTs.

In another embodiment of the compositions of the present invention, the ratio of oil to MCTs ranges from about 1:10 to about 10:1, about 1:9 to about 9:1, about 1:8 to about 8:1, about 1:7 to about 7:1, about 1:6 to about 6:1, about 1:5 to about 5:1, about 1:4 to about 4:1, about 1:3 to about 3: 1, about 1:2 to about 2: 1, or about 1:1, inclusive of all ranges and subranges therebetween. In yet another embodiment, the ratio of oil to MCTs is about 3:1.

### Phosphatides

The phosphatides suitable for use in the compositions of the invention include any phospholipid or mixture of phospholipids suitable for human consumption (e.g. pharmaceutically acceptable phospholipids), including but not limited to, phosphatidylcholine, phosphatidylethanolamine, phosphatidylinositol, and mixtures thereof. The term “phosphatides” as used herein also includes “lecithin,” which is the commercial or popular name for a mixture of naturally occurring phosphatides or phospholipids.

In one embodiment of the compositions of the present invention, one or more phosphatides are present in a unit dose in an amount of from about 10 mg to about 100 mg, from about 20 mg to about 60 mg, or from about 30 mg to about 40 mg, including about 10 mg, about 20 mg, about 30 mg, about 40 mg, about 50 mg, about 60 mg, about 70 mg, about 80 mg, about 90 mg, or about 100 mg, including all ranges and subranges therebetween. In one embodiment, the phosphatide(s) in a unit dose of the compositions of the present invention is present as about 80 mg to about 100 mg of phosphatidylcholine. In another embodiment, the phosphatide(s) in a unit dose of the compositions of the present invention is present as about 90 mg of phosphatidylcholine.

Alternatively, the concentration of the one or more phosphatides in the compositions of the present invention can be expressed in units of weight %. For example the concentration of phosphatide can range from about 0.1 wt. % to about 10 wt. %, about 0.5 wt. % to about 5 wt. %, about 1 wt. % to about 4 wt. %, and about 2 wt % to about 3.5 wt %, about 0.1 wt. %, about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, and about 10 wt. %, inclusive of all ranges and subranges therebetween.

In addition, Applicants have found that the use of perilla oil, medium chain triglycerides, and/or phosphatide(s) (e.g., phosphatidylcholine) provides an improved formulation, enhancing uptake of the Magnolia officinalis and Phellodendron amurense, while minimizing negative side effects. Although not bound by any particular theory or mechanism, it is possible that the oil, polar-fatty molecules, and/or phosphatide(s) form stabilized micelles, emulsions, droplets, or similar organization in the body, assisting uptake of the active compounds in the extracts. The result is smoother, more regulated uptake of these bioactive components. Individuals taking the improved formulations of the present invention experience the benefits of the combined Magnolia officinal and Phellodendron amurense without undesirable side effects.

### Turmeric

Turmeric is reported to have medicinal properties, including, for example, antioxidant activity. In addition, turmeric is often used as a natural coloring agent. In one embodiment of the compositions of the present invention, turmeric is used in the form of a powder. In another embodiment, turmeric is a component in a softgel capsule which comprises a composition of the present invention.

### Additional Ingredients

Non-limiting examples of suitable additional ingredients include, for example liquids, such as water or aqueous solutions (e.g., saline). Additional additives can also include auxiliary, stabilizing, thickening, lubricating and coloring agents; excipients such as starch (or starch paste), mannitol, glucose, lactose, sucrose, gelatin, malt, rice, flour, chalk, silica gel (or colloidal silica), sodium stearate, glycerol monostearate, magnesium stearate, talc, sodium chloride, dried skim milk, glycerol, propylene, glycol, water, ethanol, urea, keratin, cellulose, magnesium carbonate, and the like. Also, the compositions of the present invention can include, for example, sweetening agents such as fructose, aspartame or saccharin (sodium saccharine); flavoring agents such as peppermint, oil of wintergreen, or cherry; coloring agents; and preserving agents, to provide a pharmaceutically palatable preparation. The additional additives are of pharmaceutical grade.

Moreover, when the compositions of the present invention are prepared in the form of a tablet or pill, the compositions may be coated to delay disintegration and absorption in the gastrointestinal tract (e.g., with an enteric coating), thereby providing a sustained action over an extended period of time. A time delay material such as glycerol monostearate or glycerol stearate may also be used. The present compositions, if desired, can also contain minor amounts of wetting or emulsifying agents, or pH buffering agents.

In one embodiment, the compositions of the present invention are substantially free of citrus, monosodium glutamate, wheat, gluten, corn, zein, starch, dairy, casein, whey, milk derivatives, yeast, soy, sulfate, phosphates (other than coenzymes), and preservatives. In another embodiment, none of the ingredients in the compositions are derived from genetically modified organisms.

### Compositions of the Invention

The compositions of the invention are useful in regulating numerous disorders, including those associated with adrenal fatigue and insulin resistance, a precursor to Type 2 diabetes. Such disorders include, for example, Addison's hypoadrenalism, insomnia, chronic fatigue, depression, mood disorders, irritability, hypertension, uncontrolled or stress-related eating, weight gain, poor appetite, memory loss, and suppressed immune system.

The combination of the components of the invention can act in a synergistic way and do not exhibit the effects when administered separately.

Due to the activity of the compositions of the invention, the compositions are advantageously useful in veterinary and human medicine. The invention provides methods of regulating disorders by administration to a patient of an effective amount of a composition of the invention. The patient is a mammal, including, but not limited to, a cat, a dog or a human. In one embodiment, the patient is a human.

Various delivery systems are known, e.g., encapsulation in liposomes, microparticles, microcapsules, capsules, etc., and can be used to administer a compound of the invention. The mode of administration is left to the discretion of the practitioner, and will depend in-part upon the site of the medical condition. In most instances, administration will result in the release of at least some of the compounds of the invention into the bloodstream.

In another embodiment, the compounds of the invention can be delivered in a vesicle, for example a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In one embodiment, the compositions of the present invention are administered orally. Any conventional oral form suitable for use with the compositions of the present invention. For example, the compositions of the present invention can take the form of tablets, pills, lozenges, pellets, capsules, capsules containing liquids, powders, granules, emulsions, suspensions (oily or aqueous), solutions, syrups, elixirs or any other form suitable for oral use. In a particular embodiment, the compositions of the present invention are formulated as softgel capsules.

Other examples of suitable pharmaceutical vehicles are described in “Remington's Pharmaceutical Sciences” by E. W. Martin. In one embodiment, the compositions of the present invention are in the form of a capsule (see e.g., U.S. Pat. No. 5,698,155). In another embodiment, the compositions of the present invention are in the form of a powder. In yet another embodiment, the compositions of the present invention are in the form of an aqueous suspension or solution of a powder. In still another embodiment, the compositions of the present invention can be administered with water or juice, up to five times per day. Compounds and compositions of the invention for oral delivery can also be formulated in foods and food mixes.

In another embodiment, the compositions of the present invention are formulated in accordance with routine procedures as a nutraceutical composition adapted for oral administration to human beings.

In another embodiment, the compositions of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The present compositions will contain an effective amount of the active components of the invention. “Active components of the invention” means for example, specifically at least an extract of Rhodiola rosea and either an extract of Magnolia officinalis or an extract of Phellodendron amurense, or both in combination. The term “effective amount” in regard to the active components of the invention refers to the amount of each active component necessary to provide a clinically useful effect (e.g., preventing, regulating, reducing or ameliorating symptoms associated with a condition such as adrenal fatigue, insulin resistance, etc.).

In addition to effective amount of the active components of the invention, the compositions of the present invention can also include additional ingredients such as those described herein. Some additional ingredients, for example sweetening or flavoring agents, would not typically be considered active components of the invention because they do not provide a significant clinically useful effect in a patient, but instead mainly improve e.g. the palatability and/or stability of the formulation.

The amount of an active component of the invention will be that amount which is effective in regulating a disorder or condition disclosed herein, and will depend on the nature of the disorder or condition, and can be determined by standard techniques. The precise dose to be employed in the compositions will also depend on the route of administration, and the seriousness of the disease or disorder, and should be decided according to the judgment of the practitioner and each patient's circumstances. In vitro or in vivo assays may optionally be employed to help identify optimal dosage ranges.

Oral compositions can contain 10% to 100% by weight of the active components of the invention.

In one embodiment, the compositions of the present invention comprise an extract of Rhodiola rosea, one or more extracts selected from the group consisting of Magnolia officinalis and Phellodendron amurense, at least one oil, MCTs, and optionally one or more ingredients selected from the group consisting of one or more phosphatides, beeswax, gelatin, glycerine, water, and turmeric.

In another embodiment, the compositions of the present invention comprise about 5-20% of an extract of Rhodiola rosea, about 10-30% of combined extracts of Magnolia officinalis and Phellodendron amurense; about 20-40% of perilla oil; about 5-15% of medium chain triglycerides; and optionally one or more ingredients selected from the group consisting of phosphatidylcholine, beeswax, gelatin, glycerine, water, and turmeric, wherein the concentrations of said extracts, perilla oil, medium chain triglycerides, and optional ingredients are relative to the total weight of the composition.

In another embodiment, the compositions of the present invention comprise about 12% of an extract of Rhodiola rosea, about 20% of combined extracts of Magnolia officinalis and Phellodendron amurense; about 31% of perilla oil; about 10% of medium chain triglycerides; and optionally one or more ingredients selected from the group consisting of phosphatidylcholine, beeswax, gelatin, glycerine, water, and turmeric, wherein the concentrations of said extracts, perilla oil, medium chain triglycerides, and optional ingredients are relative to the total weight of the composition.

In a particular embodiment, the present invention encompasses a composition comprising: about 12% of an extract of Rhodiola rosea; about 20% of combined extracts of Magnolia officinalis and Phellodendron amurense (purchased as Relora®); about 31% of perilla oil; about 10% of medium chain triglycerides; about 3-4% phosphatidylcholine; about 3% beeswax; about 20% kosher gelatin; about 0.005% glycerin; about 0.008% water; and about 0.005% turmeric powder.

As illustrated by the examples below, Applicants have found that the compositions of the present invention effectively modulate cortisol levels by lowering, raising, or smoothing out irregularities, as needed by the affected individuals. In addition, Applicants have found that the unique formulation described herein enhances uptake of the botanical extracts used in the present invention while minimizing undesirable side effects. Furthermore, Applicants have found that the compositions of the present invention reduce insulin resistance and restore glucose-insulin levels to a healthy balance.

### Methods

In one embodiment, the present invention provides a method of treating a disease or condition associated with adrenal fatigue and/or insulin resistance comprising administering to a mammal a composition comprising an extract of Rhodiola rosea, and one or more extracts selected from the group consisting of Magnolia officinalis and Phellodendron amurense.

Non-limiting examples of diseases or conditions association with adrenal fatigue and/or insulin resistance include Addison's hypoadrenalism, insomnia, chronic fatigue, depression, mood disorders, irritability, hypertension, uncontrolled or stress-related eating, weight gain, poor appetite, memory loss, suppressed immune system, and Type 2 diabetes.

### Kits of the Invention

As described herein, the compositions of the present invention can be administered in any suitable dosage form, for example, an oral dosage form. For example, the individual components of the compositions of the present invention can be mixed together into a single dosage form, for example, in the form of a powder, capsule, or tablet, whereby consumption of the single dosage form provides simultaneous administration of each of the components of the composition.

Alternatively, the composition of the present invention can comprise separate unit dosage forms of one or more of the components, administered simultaneously or sequentially: for example, the extract of Rhodiola rosea can be in one dosage form, and one of the other extracts (Magnolia officinalis or Phellodendron amurense) can be in a separate dosage form; or the extract of Rhodiola rosea can be in one dosage form, and both of the extracts of Magnolia officinalis and Phellodendron amurense can be combined into a separate dosage form; or the extract of Rhodiola rosea can be combined with either of the extract Magnolia officinalis or Phellodendron amurense, and the other of the extracts can be in a separate dosage form by itself. Alternatively, each component could be provided in separate unit dosage forms, and administered sequentially or simultaneously to obtain the desired effect.

Whether administered as a single unit dosage form comprising each of the individual components of the composition of the present invention, or in two or more separate unit dosage forms which together provide each of the individual components of the composition of the present invention, the composition of the present invention can be provided in the form of a kit for regulating a condition in a mammal. In one embodiment, a kit comprising the composition of the present invention comprises a container comprising: an extract of Rhodiola rosea; one or more plant extracts selected from the group consisting of extracts of Magnolia officinalis and Phellodendron amurense; medium chain triglycerides; at least one oil; optionally one or more ingredients selected from the group consisting of a one or more phosphatides, beeswax, gelatin, glycerine, water, and turmeric; and instructions for use.

Alternatively, in another embodiment, a kit comprising the composition of the present invention comprises two or more containers, wherein said two or more containers together comprise: an extract of Rhodiola rosea; one or more plant extracts selected from the group consisting of extracts of Magnolia officinalis and Phellodendron amurense; medium chain triglycerides; at least one oil; optionally one or more ingredients selected from the group consisting of one or more phosphatides, beeswax, gelatin, glycerine, water, and turmeric; and instructions for use. Optionally associated with such container(s) can be a notice describing the manufacture or sale of compositions

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

### EXAMPLES

### Example 1

A representative composition of the present invention is shown in Table 1:

| | Amount (per ~2.5 g | |
|---|---|---|
| Ingredient | total composition) | |
| An extract of Rhodiola rosea | 300 | mg |
| (>3% rosavins + >1% salidroside) | | |
| Combined extracts of Magnolia officinalis | 500 | mg |
| and Phellodendron amurense (Relora ®)* | | |
| Perilla oil | 800 | mg |
| Medium chain triglycerides | 250 | mg |
| (from raw palm fruit) | | |
| Phosphatidylcholine | 90 | mg |
| Beeswax | 70 | mg |
| Kosher gelatin | 500 | mg |
| Glycerine | 12 | mg |
| Water | 20 | mg |
| Turmeric powder^{†} | 12 | mg |

*The extracts of Magnolia officinalis and Phellodendron amurense were purchased as Relora ® (commercially available from Next Pharmaceuticals) and incorporated into the composition of Example 1 in the purchased form.
^{†}Present in the softgel capsule

The composition of Table 1 is in the form of a softgel capsule comprising the indicated components. The recommended daily dose of the composition of Example 1 is 2 softgel capsules.

### Example 2

The composition of Example 1 was tested in a clinical study to determine its efficacy in treating diseases or conditions associated with adrenal fatigue. The 25 subjects in the study presented with symptoms of adrenal fatigue pre-treatment. The subjects were administered two softgel capsules containing the composition of Example 1 daily over a six-week period. Free cortisol and DHEA levels were measured in saliva samples using known methods, which are known to correlate with free blood (plasma) hormone levels. See, e.g., Garrison, R., Chambliss W G, Effect of a proprietary Magnolia and Phellodendron extract on weight management: a pilot, double-blind, placebo-controlled clinical trial, Alternative Therapies in Health and Medicine 2006, 12(1):50-54.

Cortisol levels were measured in each of the 25 subjects' saliva samples at four time points during the day (7:00 am, 11:00 am, 5:00 pm, and 10:00 pm) at “T₀” (before treatment with the composition of Example 1) and at “T₂” after six weeks' treatment with the composition of Example 1.

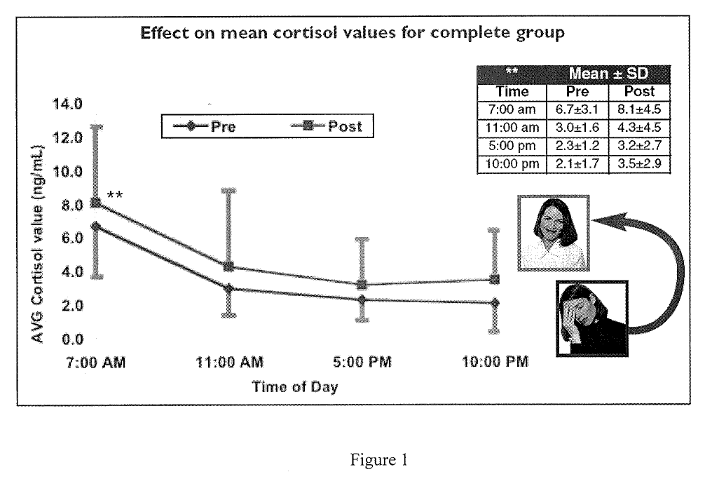

The results of the clinical study are presented in FIG. 1. The average cortisol levels of the 25 subjects' saliva samples at T₀ and T₂ are plotted as curves on a graph of cortisol concentration vs. time of day. In the legend of FIG. 1, “Pre” refers to cortisol measurements at T₀ (“pre-treatment”), and “Post” refers to cortisol measurements at T₂ (“post-treatment”). Both curves reflect the natural “rhythm” of cortisol levels, decreasing smoothly over the course of a day. But the “Post” curve shows higher cortisol levels at all time points during the day. In particular, the average first-morning cortisol level increased from a depressed value of 6.7 ng/mL (“Pre”) to a healthier level of 8.1 ng/mL. Similarly, the average late-afternoon cortisol level increased from a depressed value of 2.1 ng/mL (“Pre”) to a healthier level of 3.5 ng/mL (“Post”). The changes in average cortisol levels before and after six weeks' treatment of the composition of Example 1 were statistically significant (P<0.005 and ANOVA P<0.001). These results indicate that the cortisol levels substantially improved in the study subjects (on average) after six weeks of treatment. The results are consistent with the composition's adaptogenic effect.

### Example 3

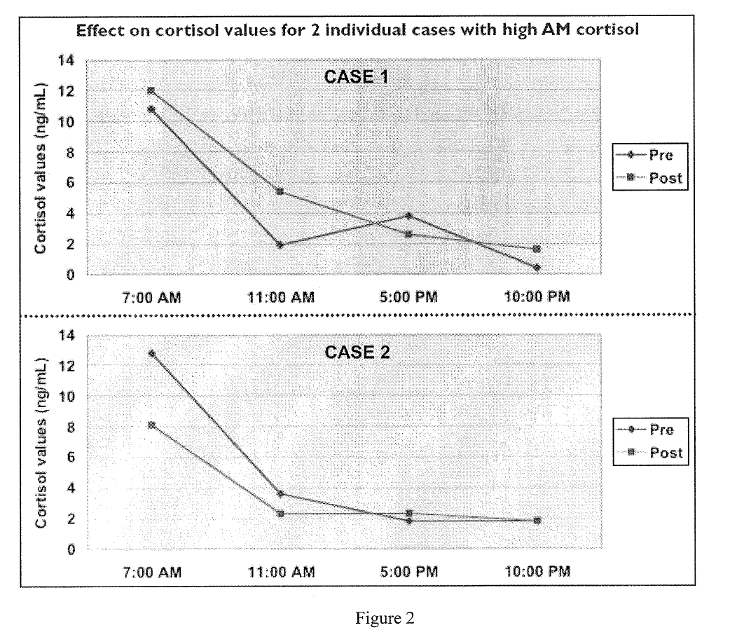

A focused analysis of the clinical study was conducted for a subgroup of subjects with elevated first-morning cortisol levels. The results for two representative subjects are shown as separate graphs (Case 1 and Case 2) in FIG. 2. As illustrated by the “Pre” curves, both subjects had higher than normal morning cortisol levels (12 ng/mL or greater at 7:00 am) and irregular cortisol “rhythm” over the course of the day. After six weeks' treatment with the composition of Example 1, the subjects' morning cortisol levels decreased, and their daily cortisol rhythms evened out, as illustrated by the “Post” curves. The results are consistent with the composition's adaptogenic effect.

An additional focused analysis was conducted for a subgroup of subjects (13) who showed lower-than-normal morning salivary cortisol levels. For these 13 subjects, six weeks' treatment with the composition of Example 1 resulted in an increase in average first-morning (7:00 am) cortisol levels from 4.2 ng/mL to 5.9 ng/mL (results not shown). Again, the results are consistent with the composition's adaptogenic effect.

### Example 4

The composition of Example 1 was tested for its effects on insulin resistance, as assessed by glucose/insulin (GI) ratio and by homeostatic model assessment (HOMA). Two individuals showing insulin resistance (GI ratio<4.5 and HOMA>4.5) were administered two softgel capsules containing the composition of Example 1 once daily for six weeks. The subjects' GI ratios and HOMA values were measured before treatment (“Pre”) and after six weeks' treatment with the composition of Example 1 (“Post”).

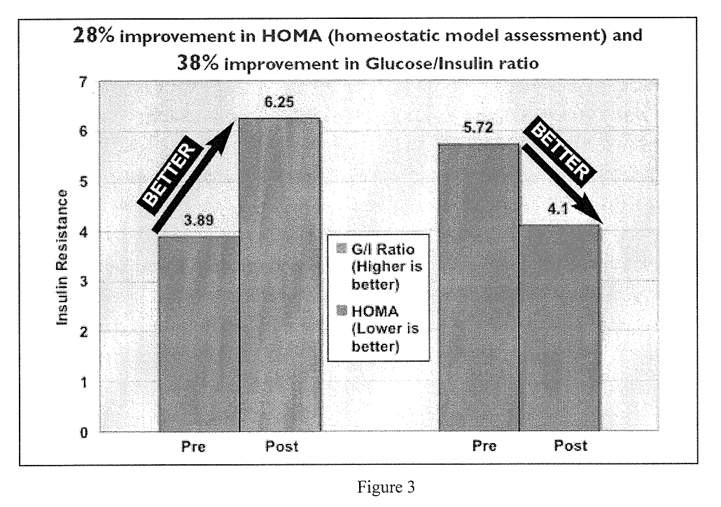

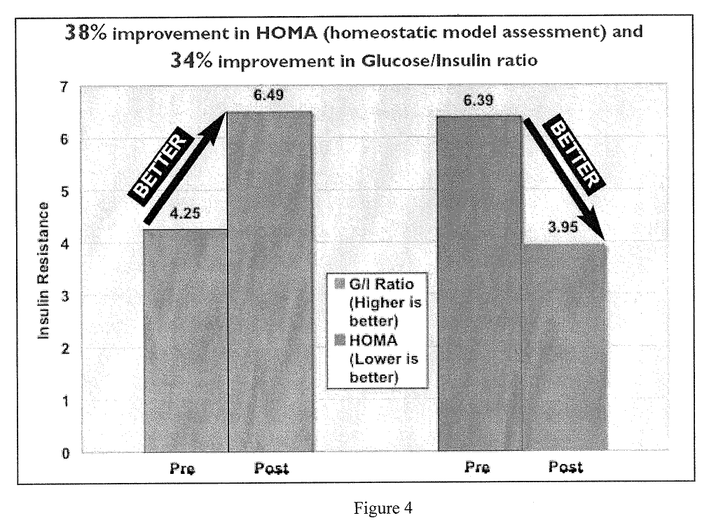

The results of the study are shown in FIG. 3 (Subject #1) and FIG. 4 (Subject #2). As illustrated by FIG. 3 for Subject #1, the GI ratio increased 38% and the HOMA value decreased 28%. As illustrated by FIG. 4 for Subject #2, the GI ratio increased 34% and the HOMA value decreased 38%. These results indicate that both subjects showed significantly improved sensitivity to insulin after six weeks' treatment with the composition of Example 1.

The results of the clinical studies also established that the benefits of the compositions of the present invention have cumulative benefits, and that the subjects did not experience negative side effects typically associated with energy stimulants (e.g., increased anxiety or jittery feelings).

Various references have been cited herein, each of which is incorporated herein by reference in its entirety for all purposes.

What is claimed:

1. A composition comprising an extract of Rhodiola rosea and one or more extracts selected from the group consisting of Magnolia officinalis and Phellodendron amurense.

2. The composition of claim 1, wherein said an extract of Rhodiola rosea is present in the amount of about 5% to about 20% by weight of said composition.

3. The composition of claim 1, wherein said an extract of Rhodiola rosea comprises one or more compounds selected from the group consisting of rosavin, rosin, rosarin, and salidroside.

4. The composition of claim 1, wherein said an extract of Rhodiola rosea comprises at least about 3% rosavins.

5. The composition of claim 1, wherein said an extract of Rhodiola rosea comprises at least about 1% salidroside.

6 The composition of claim 1, wherein said an extract of Rhodiola rosea comprises rosavins and salidroside in a ratio of about 2:1 to about 4:1.

7. The composition of claim 1, further comprising at least one oil.

8. The composition of claim 7, wherein said at least one oil comprises at least about 50% of alpha-linolenic acid.

9. The composition of claim 7, further comprising medium chain triglycerides.

10. The composition of claim 9, further comprising one or more ingredients selected from the group consisting of one or more phosphatides, beeswax, gelatin, glycerine, water, and turmeric.

11. The composition of claim 10, wherein said one or more phosphatides is selected from the group consisting of phosphatidylcholine, phosphatidylethanolamine, phosphatidylinositol, lecithin, and combinations thereof

12. The composition of claim 1, comprising an extract of Rhodiola rosea and an extract of Magnolia officinalis.

13. The composition of claim 1, comprising an extract of Rhodiola rosea and an extract of Phellodendron amurense.

14. The composition of claim 1, comprising an extract of Rhodiola rosea, an extract of Magnolia officinalis, and an extract of Phellodendron amurense.

15. The composition of claim 14, wherein the ratio by weight of said an extract of Rhodiola rosea to the combined extracts of Magnolia officinalis and Phellodendron amurense ranges from about 2:5 to about 4:5

16. The composition of claim 15, wherein said ratio is about 3:5.

17. The composition of claim 10, comprising:
a. about 5-20% of an extract of Rhodiola rosea;
b. about 10-30% of combined extracts of Magnolia officinalis and Phellodendron amurense;
c. about 20-40% of perilla oil;
d. about 5-15% of medium chain triglycerides; and
e. optionally one or more ingredients selected from the group consisting of phosphatidylcholine, beeswax, gelatin, glycerine, water, and turmeric,
wherein the concentrations of said extracts, perilla oil, medium chain triglycerides, and optional ingredients are relative to the total weight of the composition.

18. An oral dosage form comprising the composition of claim 1.

19. The dosage form of claim 18, in the form of a capsule.

20. A method of treating a disease or condition associated with adrenal fatigue and/or insulin resistance comprising administering to a mammal the composition of claim 1.

21. The method of claim 20, wherein said disease or condition is selected from the group consisting of Addison's hypoadrenalism, insomnia, chronic fatigue, depression, mood disorders, irritability, hypertension, uncontrolled or stress-related eating, weight gain, poor appetite, memory loss, suppressed immune system, and Type 2 diabetes.

22. A kit for treating a disease or condition associated with adrenal fatigue and/or insulin resistance comprising a container comprising:
a. an extract of Rhodiola rosea;
b. one or more extracts selected from the group consisting of Magnolia officinalis and Phellodendron amurense;
c. medium chain triglycerides;
d. at least one oil;
e. optionally one or more ingredients selected from the group consisting of one or more phosphatides, beeswax, gelatin, glycerine, water, and turmeric; and
f. instructions for use.
